(19) United States
(12) Patent Application Publication
(10) Pub. No.: US 2006/0089553 A1
(43) Pub. Date: Apr. 27, 2006

(54) Method and apparatus for investigating histology of epithelial tissue

(75) Inventor: Symon D'Oyly Cotton, Great Gransden, Sandy (GB)

Correspondence Address: FOGG AND ASSOCIATES, LLC, P.O. BOX 581339, MINNEAPOLIS, MN, 55458-1339, US

(73) Assignee: Astron Clinica Limited, Toft, Cambridge (GB)

(21) Appl. No.: 10/521,639

(22) PCT Filed: Jul. 21, 2003
(86) PCT No.: PCT/GB03/03245

§ 371 Date: Sep. 6, 2005

(30) Foreign Application Priority Data
Jul. 19, 2002 (GB) 0216847.4
Nov. 1, 2002 (GB) 0225444.9

(51) Int. Cl. A61B 6/00

(52) U.S. Cl. 600/473

(57) ABSTRACT

A method for monitoring the presence of selected chromophores in a sample of epithelial tissue, independent of the amount of a predetermined chromophore, the method comprising: illuminating an area of tissue by projecting light from a light source of at least two different wavelengths λ₁, λ₂; receiving light remitted by the illuminated area of tissue at a photoreceptor; analysing the received light to identify and measure the proportion of light of each wavelength remitted from the tissue Iᵣ (λ); calculating the ratio of light at each wavelength returned from the tissue Rₜ, (λ), and then calculating Z=Formula (I); where 1 is chosen such that Z is independent of the amount of predetermined chromophore. Typically 1 is calculated such that Z=Formula (II); where j and k are such that 2j α(λ₁)=2kj α(λ₂)=1 where α(λ₁) and α(λ₂) are the absorbtion coefficients for the predetermined chromophore at each wavelength.

### FIELD OF THE INVENTION

The present invention relates to a method and apparatus for investigating the histology of epithelial tissue to provide an analysis of the tissue which is independent of the amount of a chosen chromophore, such as melanin or haemoglobin. The invention is applicable with particular advantage for investigating skin histology for the investigation of skin cancers.

Non-melanoma skin cancer accounts for 90% of skin cancers. Within the grouping of non melanoma skin cancer there are two predominant forms Basal Cell Carcinoma (BCC) and Squamous Cell Carcinoma (SCC) with approximately 75% being BCC's and 20% being SCC's: indeed, BCC is not only the most common form of skin cancer, it is also the most common form of cancer in humans; it is estimated 1 in 3 Americans will develop a BCC during their life time.

Both forms of cancer are believed to be linked to Ultra Violet exposure causing damage to the DNA of cells existing within the upper layers of the skin. The cancers typically cause local destruction of tissue, but although they have the power to metastasise, the percentage chance of metastasis is far lower than for melanoma, the more aggressive form of skin cancer.

A large number of different treatment options are now available for non-melanoma skin cancer ranging from surgical excision to light activated drugs that destroy the tumour, to locally applied cryotherapy. The decision on which treatment option is the most suitable depends largely on at which stage the cancer is in its life cycle and the site of the tumour. Both BCC's and SCC's begin life with the tumour cells confined to solely to the epidermis—SCC's are commonly called Actinic Keratosis at this stage—a stage at which they are histologically referred to as “superficial”. The cancer can then penetrate and populate the dermis at which point a histologist would refer to them as “infiltrating” or “invasive”. Non-surgical treatment has been shown to be effective for treating superficial cancers but is far less effective for infiltrating or invasive cases when surgery is the best option. There are many reasons to prefer a non-surgical intervention namely a better cosmetic result is often achieved and the treatment can be applied at a primary care level—something which is important when the large numbers of these cancers are considered. However, it is also not desirable to treat invasive non-melanoma cancer in such a manner as there is a possibility that not all the cancer will be destroyed therefore requiring surgery at a later stage.

Currently, there is no reliable method available to assess whether such a cancer is superficial, that can be applied widely enough to reach practising dermatologists and general practice. Confocal microscopy can be used to view the malignant cells and indeed assess whether they are intra-epidermal or not but both the high cost and time required to assess a patient have so far confined its use to research institutions. A useful tool would therefore be one that is both effective in distinguishing superficial from infiltrating and invasive non-melanoma skin cancer and which is also applicable to a primary care setting.

Skin can be considered to be a layered structure with the epidermis lying over the dermis. The junction between the two layers is called the dermo-epidermal junction and anchored to this layer are cells called melanocytes that produce the pigment melanin. It is these melanocytes which dictate the colour of our skin with black skin having the same number of melanocytes as white skin but the production of melanin being higher. The melanin produced is taken up by keratinocytes in the epidermis which migrate to the surface before flaking and being discarded. The dermis, in contrast, is formed largely from collagen fibres which are tightly bound together and blood vessels.

It has been found that the structure of tissue can be analysed to investigate the presence of chromophores in the tissue by illuminating the tissue with light and then analysing the proportion of light remitted by the tissue. Examples are described in our previously published applications WO98/22023 and WO00/75637. Optically both layers exhibit markedly different properties most notably in the amount to which they scatter light. The epidermis is a low scattering regime in contrast to the dermis where the collagen fibres are on a comparable scale with the wavelengths of visible and near infrared light resulting in a strong interaction and high scattering.

Light striking the outer layer of the skin therefore first has to traverse the epidermis suffering absorption from any pigments, typically melanin, being present. The low scattering nature of the epidermis will ensure that any remaining light enters the dermis with absorption occurring from the collagen fibres and any haemoglobin present. The high scattering nature of the dermis will then return a proportion back into the epidermis which it will travel through again before being remitted from the tissue.

### SUMMARY OF THE INVENTION

According to the invention there is provided a method for monitoring the presence of selected chromophores in a sample of epithelial tissue, independent of the amount of a predetermined chromophore, the method comprising:
illuminating an area of tissue by projecting light of at least two different wavelengths, λ₁, λ₂ from a light source;
receiving light remitted by the illuminated area of tissue at a photoreceptor; analysing the received light to obtain a measurement Rₜ(λ) for each wavelength and then calculating:

$$Z = \frac{{R}_{t} ( {λ}_{1} )}{{{R}_{t} ( {λ}_{2} )}^{l}}$$

where ι is chosen such that Z is independent of the amount of predetermined chromophore.

According to a further aspect of the invention there is provided a method of forming an image of an area of epithelial tissue independent of the amount of a predetermined chromophore in the tissue, locations, formed by obtaining Z for a plurality of locations within the area, Z being obtained by illuminating an area of tissue by projecting light of at least two different wavelengths λ₁, λ₂ from a light source;
receiving light remitted by the illuminated area of tissue at a photoreceptor;
analysing the received light to obtain a measurement Rₜ(λ) for each wavelength and then calculating:

$$Z = \frac{{R}_{t} ( {λ}_{1} )}{{{R}_{t} ( {λ}_{2} )}^{l}}$$

where ι is chosen such that Z is independent of the amount of predetermined chromophore.

Rₜ(λ) could be the signal measured by an instrument/camera as any scaling or intensity constant could would cancel out or be taken account of through the choice of ι. Preferably however, Rₜ(λ) is calculated by analysing the received light to identify and measure the proportion of light of each wavelength remitted from the tissue Iᵣ(λ); and calculating the ratio of light at each wavelength returned from the tissue Rₜ(λ).

As will be described and mathematically proved further in the specification, for each pair of wavelengths λ₁, λ₂ and predetermined chromophore, a value ι exists where Z is independent of the presence of the amount of predetermined chromophore. This could be found by the skilled addressee by trial and error, especially if a series of such Z values are calculated and mapped. An experienced and skilled reader of such mapped images could from his own experience identify those Z images which are independent of any given chromophore.

However, the value ι may be calculated by using the fact that for any pair of wavelengths λ₁, λ₂ and chromophore, there exists constants j and k such that 2jα(λ₁)=2kjα(λ₂)=1 where α(λ₁) and α(λ₂) are the absorbtion coefficients for the predetermined chromophore at each wavelength and

$$Z = \frac{{{R}_{d} ( c , h , {λ}_{1} )}^{j}}{{{R}_{d} ( c , h , {λ}_{2} )}^{jk}} = \frac{{{R}_{t} ( {λ}_{1} )}^{j}}{{{R}_{t} ( {λ}_{2} )}^{jk}} = \frac{{R}_{t} ( {λ}_{1} )}{{{R}_{t} ( {λ}_{2} )}^{l}} .$$

The benefits of this measurement technique are that measurements at just 2 wavelengths are required, the calculation is simple, the method is tolerant of measurement noise and calibration errors, it eliminates the effects of a predetermined chromophore which is a major absorber in the epithelial tissue, and it is sensitive to small differences in collagen.

Examples of particular chromophores whose presence may be monitored include: melanin, blood, haemoglobin, oxy-haemoglobin, bilirubin, tattoo pigments and dyestuffs, keratin, collagen and hair. There are occasions where an image of epithelial tissue independent upon the amount of any of these chromophores would be medically extremely useful and thus any of these chromophores or indeed others may be chosen as the ‘predetermined chromophore’. Measurements which ‘ignore’ the melanin level or the blood/haemoglobin level in the tissue, can be extremely useful in identifying BCC and SCC in the skin. However in babies, being able to provide a measurement independent of the amount of bilirubin in the tissue can be useful.

The invention is applicable to the investigation of any epithelial tissue, such as skin and linings of the respiratory and digestive tracts, the cervix and other surfaces to which visual access may be had, such as the retina. Clearly for many of the tissues, taking the required measurements would require the use of an endoscope—the use of which would be apparent to the skilled addressee of the specification.

The invention also provides apparatus for analysing skin in accordance with this method. There are several such apparatus available—for illuminating tissue with light of a given wavelength, measuring light remitted by the tissue and then analysing the resultant remitted light to provide the Z value. Such apparatus may then be coupled to an imaging device to provide a visual image representative of the level of selected chromophores in the tissue.

The mathematics of the operation can be analysed with reference to the level of melanin in skin as an example. As will be apparent to the skilled addressee of the specification, these formulae apply in relation to any other chromophore and its presence in epithelial tissue. If the light striking the tissue is described as I₀(λ) where λ refers to the wavelength of light, absorption due to melanin as A(m,λ) where m refers to the amount of melanin present and the proportion returned from the dermis as R_{d}(c,h,λ), where c relates to the amount of collagen present and h haemoglobin: Iᵣ(λ), that proportion of light remitted from the skin can be described as Iᵣ(λ)=I₀(λ)A(m,λ)²R_{d}(c,h,λ). The A(m,λ)² term is due to light traversing the epidermis twice. The absorption of light by melanin A(m,λ) can be shown to be an exponential term of the from e^{mα(λ)} where α is the absorption coefficient of melanin therefore resulting in:
Iᵣ(λ)=I₀(λ)e^{2mα(λ)}R_{d}(c,h,λ).
And

$${R}_{t} ( λ ) = \frac{{I}_{r} ( λ )}{{I}_{0} ( λ )} = {ⅇ}^{2 m α ( λ )} {R}_{d} ( c , h , λ )$$

the ratio of light returned from the tissue if Rₜ(λ) is computed at different wavelengths and then divided by one another G(λ₁,λ₂) can be found where

$$G ( {λ}_{1} , {λ}_{2} ) = \frac{{ⅇ}^{2 m α ( {λ}_{1} )} {R}_{d} ( c , h , {λ}_{1} )}{{ⅇ}^{2 m α ( {λ}_{2} )} {R}_{d} ( c , h , {λ}_{2} )}$$

α(λ₁) and α(λ₂) are constants if λ₁ and λ₂ are fixed so there exist a series of constants j and k where 2jα(λ₁)=2kjα(λ₂)=1 therefore there exists Z where

$$Z ( {λ}_{1} , {λ}_{2} ) = \frac{{ⅇ}^{2 mj α ( {λ}_{1} )} {{R}_{d} ( c , h , {λ}_{1} )}^{j}}{{ⅇ}^{2 mjk α ( {λ}_{2} )} {{R}_{d} ( c , h , {λ}_{2} )}^{jk}} = \frac{{ⅇ}^{m} {{R}_{d} ( c , h , {λ}_{1} )}^{j}}{{ⅇ}^{m} {{R}_{d} ( c , h , {λ}_{2} )}^{jk}} = \frac{{{R}_{d} ( c , h , {λ}_{1} )}^{j}}{{{R}_{d} ( c , h , {λ}_{2} )}^{jk}}$$

and therefore

$$Z = \frac{{{R}_{d} ( c , h , {λ}_{1} )}^{j}}{{{R}_{d} ( c , h , {λ}_{2} )}^{jk}} = \frac{{{R}_{t} ( {λ}_{1} )}^{j}}{{{R}_{t} ( {λ}_{2} )}^{jk}} = \frac{{R}_{t} ( {λ}_{1} )}{{{R}_{t} ( {λ}_{2} )}^{l}}$$

R₁(λ₁) and Rₜ(λ₂) are straightforward to measure and j and k can easily be calculated by considering the absorption properties of melanin against wavelength or by experiment. From the terms j and k, ι can readily be calculated. The resulting term Z is independent of the melanin term being constructed solely from differences in the dermal component R_{d}. If wavelengths are then chosen where the haemoglobin term, h, is very small Z then becomes purely dependent on non-haemoglobin changes to the dermal component such as collagen and the presence of any other interesting material. Such wavelengths are easily accessible by silicon based sensors above approximately 600 nm. It should therefore be possible construct images showing the variation of Z which may carry information pertinent to the structure of a skin lesion and in particular a BCC or SCC.

Images to be constructed, typically comprise in the region of 700 pixels per cm, to give suitable resolution. However, it will be appreciated that there will be times when greater resolution is required to study a condition correctly—or there may be occasions where less resolution is possible/desirable.

Typically the image will be post processed based on frequency analysis and local contrast enhancement.

It will be appreciated by the skilled addressee that for any two wavelengths and predetermined chromophore, there will exist j and k for which 2jα(λ₁)=2kjα(λ₁)=1 where α(λ₁) and α(λ₂ ) are the absorbtion coefficients for the predetermined chromophore at each wavelength. Thus for different j and k, Z values independent of various chromophores can be calculated. Thus

$$Z = \frac{{R}_{t} ( {λ}_{1} )}{{{R}_{t} ( {λ}_{2} )}^{l}}$$

can be calculated using different values of ι for different predetermined chromophores.

Although any pair of wavelengths may be used, preferably there is a difference in change in absorbtion for each wavelengh caused by changes in collagen level. Also it has been found that wavelengths with a difference of more than 200 nm give effective results.

There will also be cases where light of more than two wavelengths are used to illuminate the tissue. With three wavelengths, there will be three pairs of wavelengths and calculations which can be made with the three different corresponding j,k and ι values to provide greater accuracy in the calculations of Z at particular points.

To test this hypothesis images of BCC's were acquired from 10 lesions including 5 superficial and 5 infiltrating/invasive. The wavelengths used included 700 nm and 940 nm at which the absorption of haemoglobin is negligible Z was then computed across each lesion where the predetermined chromophore is melanin and thus Z is independent of the amount of melanin in the tissue studied.

Two examples are shown in the accompanying figures, in which:

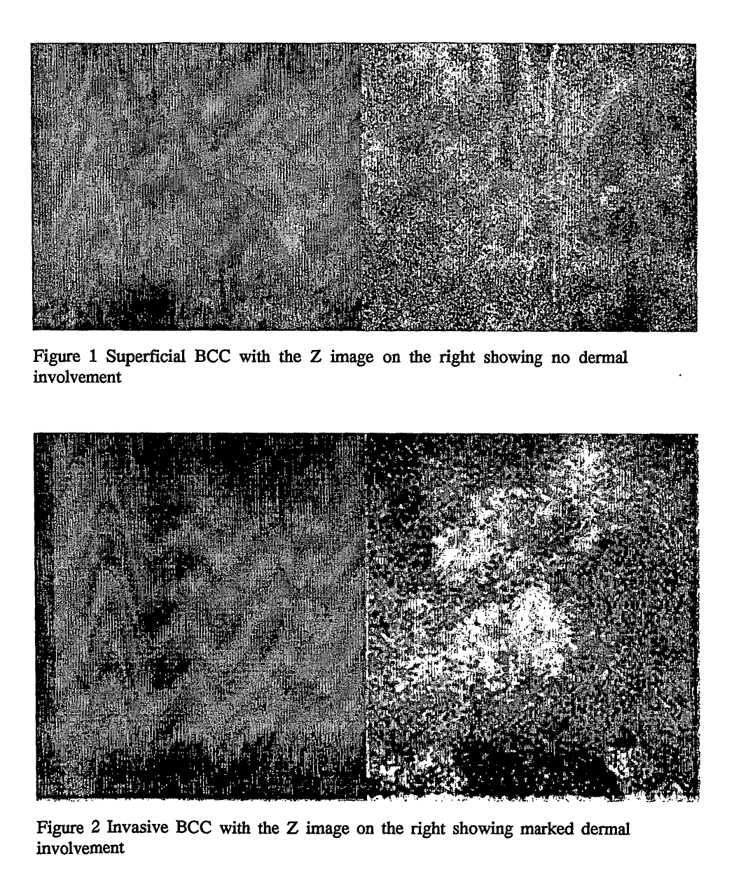

FIG. 1 shows a histologically confirmed superficial BCC with the Z image to the right. The Z image shows little difference between the surrounding tissue and the BCC.; which indicates little dermal involvement.

In contrast FIG. 2 shows an invasive BCC with its Z image ( on the right hand side) indicating a marked difference from the surrounding tissue indicating dermal involvement; and,

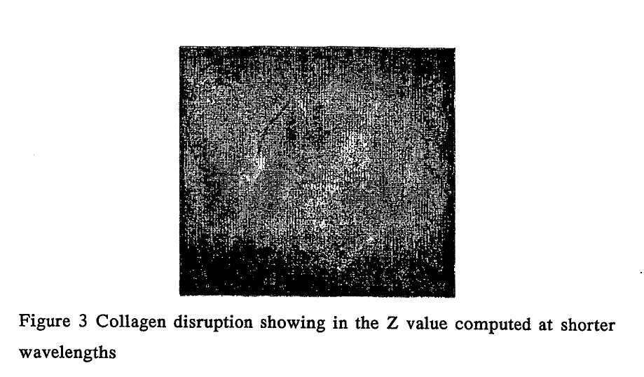

FIG. 3 below shows an example computed at these shorter wavelengths showing the extent of collagen disruption

This pattern replicated itself through out all ten lesions with the invasive and infiltrating BCC's showing deviations on the Z image compared with the surrounding tissue whilst the superficial BCC's showed no such deviation.

The Z image construction and analysis produced information able to separate superficial from infiltrating and invasive BCC's. This information is important in the management of the most common form of cancer in human's allowing a clinician to treat superficial BCC's quickly and simply without surgery whilst ensuring that those that require surgery undergo a procedure with minimum delay. Another important consideration is that the technology required to implement this technique is readily available in the form of CCD and CMOS digital cameras although controlled illumination at specific wavelengths is required. This study only examined BCC's but it is a reasonable, although untested, hypothesis that a similar approach may yield information in the case of SCC's.

The analysis in this document specifically utilized near infrared wavelengths where the absorption of haemoglobin is low. This however limits the resolution of information relating to the disruption of collagen due to the cancer, if a lower frequency is used—for instance blue and green light—the spatial resolution of the collagen increases although there is artefact due to cross over with haemoglobin. This increase in resolution however appears to allow good discrimination of the edge of the cancer, something which is important in planning surgery, particularly Mohs surgery.

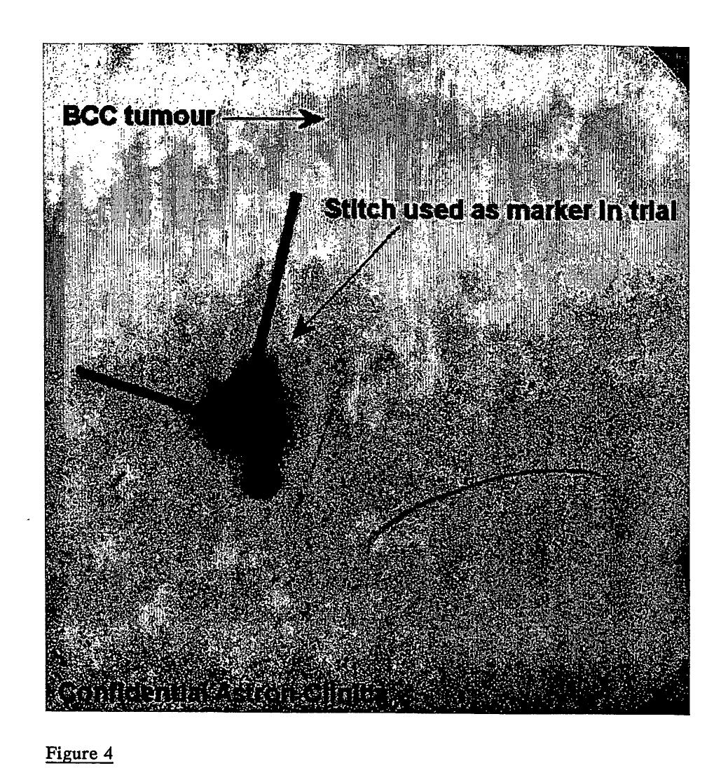

FIG. 4 shows an image of skin where a surgeon placed a stitch at the clinically observed edge of a BCC tumour—above the stitch as shown in FIG. 4. As can be seen the Z image shown (which is independent of the amount of melanin in the skin) clearly shows a difference in image of the healthy skin and the skin overlying the tumour.

1. A method for monitoring the presence of selected chromophores in a sample of epithelial tissue, independent of the amount of a predetermined chromophore, the method comprising:
illuminating an area of tissue by projecting light from a light source of at least two different wavelengths λ₁, λ₂;
receiving light remitted by the illuminated area of tissue at a photoreceptor; analysing the received light to obtain a measurement Rₜ(λ) for each wavelength and then calculating:
$$Z = \frac{{R}_{t} ( {λ}_{1} )}{{{R}_{t} ( {λ}_{2} )}^{l}}$$
where l is chosen such that Z is independent of the amount of predetermined chromophore.

2. A method according to claim 1, in which Rₜ(λ) is calculated by analysing the received light to identify and measure the proportion of light of each wavelength remitted from the tissue Iᵣ(λ); and calculating the ratio of light at each wavelength returned from the tissue Rₜ(λ).

3. A method according to claim 1, in which l is calculated such that
$$Z = \frac{{{R}_{t} ( c , h , {λ}_{1} )}^{j}}{{{R}_{t} ( c , h , {λ}_{2} )}^{jk}} = \frac{{{R}_{t} ( {λ}_{1} )}^{j}}{{{R}_{t} ( {λ}_{2} )}^{jk}} = \frac{{R}_{t} ( {λ}_{1} )}{{{R}_{t} ( {λ}_{2} )}^{l}}$$
where j and k are such that:
2jα(λ₁)=2kjα(λ₂)=1 where α(λ₁) and α(λ₂) are the absorbtion coefficients for the predetermined chromophore at each wavelength.

4. A method according to claims claim 1, in which the predetermined chromophore is melanin.

5. A method according to claim 1, in which the predetermined chromophore is haemoglobin.

6. A method according to claim 1, in which the epithelial tissue is skin.

7. A method according to claim 1, in which the wavelengths λ₁, λ₂ are chosen such that a change in collagen level causes a relatively small change in the absorbtion of λ₁, and a relatively large change in the absorbtion of λ₂.

8. A method according to claim 7, in which the difference between the two wavelengths λ₁, λ₂ is at least 200 nm.

9. A method according to claim 8, in which the wavelengths are substantially 700 nm and 940 nm respectively.

10. A method of forming an image of an area of epithelial tissue independent of the amount of a predetermined chromophore in the tissue, locations, formed by obtaining Z for a plurality of locations within the area, Z being obtained by illuminating an area of tissue by projecting light from a light source of at least two different wavelengths λ₁, λ₂;
receiving light remitted by the illuminated area of tissue at a photoreceptor;
analysing the received light to analysing the received light to obtain a measurement Rₜ(λ) for each wavelength and then calculating:
$$Z = \frac{{R}_{t} ( {λ}_{1} )}{{{R}_{t} ( {λ}_{2} )}^{l}}$$
where l is chosen such that Z is independent of the amount of predetermined chromophore; and mapping the amounts Z at positions indicative of the location within the area of the measurement.

11. A method according to claim 10, in which Rₜ(λ) is calculated by analysing the received light to identify and measure the proportion of light of each wavelength remitted from the tissue Iᵣ(λ); and calculating the ratio of light at each wavelength returned from the tissue Rₜ(λ).

12. A method according to claim 10, in which l is calculated such that
$$Z = \frac{{{R}_{d} ( c , h , {λ}_{1} )}^{j}}{{{R}_{d} ( c , h , {λ}_{2} )}^{jk}} = \frac{{{R}_{t} ( {λ}_{1} )}^{j}}{{{R}_{t} ( {λ}_{2} )}^{jk}} = \frac{{R}_{t} ( {λ}_{1} )}{{{R}_{t} ( {λ}_{1} )}^{l}}$$
where j and k are such that 2jα(λ₁)=2kjα(λ₂)=1 where α(λ₁) and α(λ₂) are the absorbtion coefficients for the predetermined chromophore at each wavelength.

13. A method according to claim 10, in which the at least two sets of calculations
$$Z = \frac{{R}_{t} ( {λ}_{1} )}{{{R}_{t} ( {λ}_{2} )}^{l}}$$
are carried out, a first calculation with l, such that Z is independent of the amount of a first predetermined chromophore, and a second calculation with l₂ such that Z is independent of the amount of a second predetermined chromophore.

14. A method according to claim 10 in which the light source used to illuminate the tissue, is of at least three wavelengths, λ₁,λ₂,λ₃ and at least three pairs of calculations of Z are made, namely
$$Z = \frac{{R}_{t} ( {λ}_{1} )}{{{R}_{t} ( {λ}_{2} )}^{l1}} , Z = \frac{{R}_{t} ( {λ}_{2} )}{{{R}_{t} ( {λ}_{3} )}^{l2}} , Z = \frac{{R}_{t} ( {λ}_{1} )}{{{R}_{t} ( {λ}_{3} )}^{l3}}$$
where l₁ l₂ l₃ are each chosen such that Z is independent of the amount of the predetermined chromophore for the respective pair of wavelengths.

15. Apparatus for monitoring the presence of selected chromophores in a sample of epithelial tissue, independent of the amount of a predetermined chromophore comprising a light source for illuminating tissue with light of at least two different wavelengths λ₁, λ₂.
a photoreceptor for receiving images remitted by the illuminated area of tissue at a photoreceptor; and
microprocessor means for analysing the received light to identify and measure the proportion of light of each wavelength remitted from the tissue Iᵣ(λ);
calculating the ratio of light at each wavelength returned from the tissue Rₜ(λ), and then calculating:
$$Z = \frac{{R}_{t} ( {λ}_{1} )}{{{R}_{t} ( {λ}_{2} )}^{l}}$$
where l is chosen such that Z is independent of the amount of predetermined chromophore.

16. Apparatus according to claim 15, also comprising image creation means for receiving a plurality of values of Z, each for a specified location on the tissue, and providing a mapped image representing the value of Z at the plurality of locations on the tissue.
